(19) United States
(12) Patent Application Publication
(10) Pub. No.: US 2013/0266665 A1
(43) Pub. Date: Oct. 10, 2013

(54) NANOMECHANICAL BIOMARKERS FOR DISEASE THERAPY

(71) Applicants: Oliver DORIG, Los Angeles, CA (US); James Gimzewski, Los Angeles, CA (US); Jianyu Rao, Culver City, CA (US); Shivani Sharma, Los Angeles, CA (US); Chintda Santiskulvong, Monterey Park, CA (US)

(72) Inventors: Oliver DORIG, Los Angeles, CA (US); James Gimzewski, Los Angeles, CA (US); Jianyu Rao, Culver City, CA (US); Shivani Sharma, Los Angeles, CA (US); Chintda Santiskulvong, Monterey Park, CA (US)

(21) Appl. No.: 13/802,080

(22) Filed: Mar. 13, 2013

Related U.S. Application Data
(60) Provisional application No. 61/614,339, filed on Mar. 22, 2012.

(51) Int. Cl. G01N 33/50

(52) U.S. Cl. CPC G01N 33/5044
USPC 424/649; 435/29; 424/729; 514/449

(57) ABSTRACT

A method of treating a patient having cancer includes: (1) providing a biological sample from the patient, the biological sample including multiple cells; (2) detecting a response of the biological sample to a probing element; (3) based on the response, determining test values for the biological sample, the test values being indicative of a nanomechanical characteristic of the cells; (4) deriving a test nanomechanical profile characterizing a distribution of the test values; and (5) based on the test nanomechanical profile, selecting a therapeutic agent to treat the patient.

### CROSS-REFERENCE TO RELATED APPLICATION

This application claims the benefit of U.S. Provisional Application No. 61/614,339 filed on Mar. 22, 2012, the disclosure of which is incorporated herein by reference in its entirety.

### FIELD OF THE INVENTION

The invention generally relates to the analysis of nanomechanical characteristics of cells. More particularly, the invention relates to such analysis to predict sensitivity to therapeutic agents and monitor effectiveness of therapeutic agents.

### BACKGROUND

Ovarian cancer is the most lethal of the gynecological malignancies, the fifth leading cause of cancer-related deaths among women in the United States and responsible for over 13,000 deaths in 2010. The current treatment strategy for ovarian cancer is surgical removal of the tumor tissue followed by chemotherapy. For epithelial ovarian cancer, first-line chemotherapy usually involves a combination of platinum and taxane agents. Ovarian cancer is a chemotherapy-sensitive disease, but the presence of primary platinum resistance disease in about 15% of all patients and the development of platinum-resistant disease in recurrent ovarian cancer present a major therapeutic challenge. Resistance to a therapeutic agent is a cellular phenomenon indicating the inability to obtain cytotoxicity at physiologically achievable concentrations of the therapeutic agent. Effective biomarkers are lacking to predict sensitivity to therapeutic agents for ovarian cancer as well as other types of cancer.

It is against this background that a need arose to develop the nanomechanical analysis and related systems and methods described herein.

### SUMMARY

One aspect of this disclosure relates to a method of treating a patient having cancer. In one embodiment, the method includes: (1) providing a biological sample from the patient, the biological sample including multiple cells; (2) detecting a response of the biological sample to a probing element; (3) based on the response, determining test values for the biological sample, the test values being indicative of a nanomechanical characteristic of the cells; (4) deriving a test nanomechanical profile characterizing a distribution of the test values; and (5) based on the test nanomechanical profile, selecting a therapeutic agent to treat the patient.

In another embodiment, the method includes: (1) providing a first biological sample from the patient prior to administering a therapeutic agent; (2) deriving a baseline nanomechanical profile characterizing a distribution of Young's modulus values of a first set of cancerous cells in the first biological sample; (3) providing a second biological sample from the patient subsequent to administering the therapeutic agent; (4) deriving a post-treatment nanomechanical profile characterizing a distribution of Young's modulus values of a second set of cancerous cells in the second biological sample; and (5) based on a comparison between the post-treatment nanomechanical profile and the baseline nanomechanical profile, adjusting administering of the therapeutic agent to the patient.

Another aspect of this disclosure relates to a non-transitory computer-readable storage medium to monitor treatment of a patient having cancer. In one embodiment, the medium includes executable instructions to: (1) derive a baseline nanomechanical profile characterizing a distribution of Young's modulus values of a first set of cancerous cells, the first set of cancerous cells being collected from a human patient prior to administering a therapeutic agent; (2) derive a post-treatment nanomechanical profile characterizing a distribution of Young's modulus values of a second set of cancerous cells, the second set of cancerous cells being collected from the human patient subsequent to administering the therapeutic agent; and (3) based on a comparison between the post-treatment nanomechanical profile and the baseline nanomechanical profile, produce an indication of effectiveness of the therapeutic agent for the human patient.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

For a better understanding of the nature and objects of some embodiments of this disclosure, reference should be made to the following detailed description taken in conjunction with the accompanying drawings.

FIG. 1: A nanomechanical analysis system implemented in accordance with an embodiment of this disclosure.

FIG. 2A and FIG. 2B: Operation of the system of FIG. 1.

FIG. 3A and FIG. 3B: Distributions of Young's modulus values characterized according to an embodiment of this disclosure.

FIG. 4: Cisplatin-resistant SKOV3-CisR and OVCAR5-CisR cells are stiffer compared to cisplatin-sensitive SKOV3 and OVCAR5 cells. Histograms show counts versus Young's modulus values (kPa) with Gaussian fits. (A) Young's modulus for SKOV3 cells, which are softer (P<0.05) than (B) SKOV3-CisR cells. (C) OVCAR5 cells are softer (P<0.05) compared to (D) OVCAR5-CisR cells.

FIG. 5: Cisplatin induces concentration-dependent increase in the Young's modulus of OVCAR5 cells. (A) Young's modulus distribution of OVCAR5 cells showing normal distribution, (B) OCVAR5_{cisplatin 1 μg/mL} cells, and (C) OCVAR5_{cisplatin 5 μg/mL} cells. Treated cells (B, C) show significant shift toward higher stiffness (P<0.05) and bimodal distribution of cell population modulus. (D) Localized cell stiffness showing peak indentation depth for OVCAR5, (E) OCVAR5_{cisplatin 1 μg/mL}, and (F) OCVAR5_{cisplatin 5 μg/mL} cells. For an applied force of about 1 nN, an AFM tip indents about twice as much as for untreated cells (D) compared to cells after cisplatin treatment (E, F). Insets in D and F display schematics of higher and lower AFM tip indentation, respectively.

FIG. 6: Cisplatin treatment does not result in noticeable increase in the Young's modulus of OVCAR5-CisR cells. (A) Young's modulus distribution of OVCAR5-CisR and (B) OCVAR5-CisR_{cisplatin 5 μg/mL} cells. (C) Localized cell stiffness showing peak indentation depth for OVCAR5-CisR and (D) OCVAR5-CisR_{cisplatin 5 μg/mL} cells.

FIG. 7: Cisplatin-induced increase in cell stiffness of OVCAR5 cells is mediated via actin cytoskeleton. Effect of cytochalasin D treatment alone (A, D) and in combination in cisplatin on OVCAR5 (B, C) and OVCAR5-CisR (E, F) Young's modulus was observed. The histograms show results for (A) OVCAR5_{cytoD}, (B) OVCAR5_{cytOD+cisplatin}, (C) OVCAR5_{cisplatin+cytoD}, (D) OVCAR5-CiSR_{cytoD}, (E) OVCAR5R_{cytoD+cisplatin}, and (F) OVCAR5_{cisplatin+cytoD}, and indicate that cisplatin-induced increase in cell stiffness can be reversed by disrupting the actin cytoskeleton.

FIG. 8: Summary of Atomic Force Microscope-based biomechanical profiles of cisplatin-sensitive and cisplatin-resistant ovarian cancer cells.

FIG. 9: Cisplatin-sensitive (A-C) and resistant cells (D-F) possess significantly different subcellular actin cytoskeleton structure and organization as revealed via Stimulated Emission Depletion microscopy. (A) Typical actin cytoskeleton in OVCAR5 entire cell. (B) Zoom-in view showing short bundled (Δ) actin filaments. (C) Scattered, randomly oriented actin fibers within the cytoplasmic region as aggregates (*), intertwined (**), or short (***) filaments. (D) OVCAR5-CisR cell with radially aligned long actin filaments extending from nucleus up to the cell membrane (#), seen at higher resolution in zoom-in view (E). (F) An example of actin-mediated extensions of the cell forming cell-cell contacts (# #) in resistant cells. Nuclear regions based on differential interference contrast images are marked with twisted arrows in A and D.

FIG. 10: Determination of a Stimulated Emission Depletion microscope's resolution (A) images of crimson beads filled with Atto647N. The resolution on the image has been quantified in (B). (B) Line profiles of beads (marked in image (A) show full width at half maximum of about 72.3 nm. Stimulated Emission Depletion microscopy more clearly revealed OVCAR5 cell actin structural organization compared to confocal imaging. (C) Differential Interference Contrast image of OVCAR5 cell nuclear region and (D) corresponding confocal image of F-actin. (E) Differential Interference Contrast image of cell nuclear region and (F) corresponding Stimulated Emission Depletion image showing actin filaments. The inset shows a zoomed in section of actin fibers not resolved via confocal imaging.

FIG. 11: Stimulated Emission Depletion images showing OVCAR5 F-actin organization from top to bottom region (A-D) of a cell. The actin fibers envelope around the cell nucleus (central unstained region) and are not directionally oriented. No stress fibers are observed at the base of the nucleus.

### DETAILED DESCRIPTION

### Definitions

The following definitions apply to some of the aspects described with respect to some embodiments of this disclosure. These definitions may likewise be expanded upon herein.

As used herein, the singular terms “a,” “an,” and “the” include plural referents unless the context clearly dictates otherwise. Thus, for example, reference to an object can include multiple objects unless the context clearly dictates otherwise.

As used herein, the term “set” refers to a collection of one or more objects. Thus, for example, a set of objects can include a single object or multiple objects. Objects of a set also can be referred to as members of the set. Objects of a set can be the same or different. In some instances, objects of a set can share one or more common characteristics.

As used herein, the term “adjacent” refers to being near or adjoining Adjacent objects can be spaced apart from one another or can be in actual or direct contact with one another. In some instances, adjacent objects can be connected to one another or can be formed integrally with one another.

As used herein, the terms “inner,” “outer,” “upper,” “upwardly,” “lower,” “downwardly,” “lateral,” and “laterally” refer to a relative orientation of a set of objects, such as in accordance with the drawings, but do not require a particular orientation of those objects during manufacturing or use.

As used herein, the terms “connect,” “connected,” and “connection” refer to an operational coupling or linking Connected objects can be directly coupled to one another or can be indirectly coupled to one another, such as via another set of objects.

As used herein, the terms “substantially” and “substantial” refer to a considerable degree or extent. When used in conjunction with an event or circumstance, the terms can refer to instances in which the event or circumstance occurs precisely as well as instances in which the event or circumstance occurs to a close approximation, such as accounting for typical tolerance levels or variability of the embodiments described herein.

As used herein with reference to a cell, the term “membrane” refers to a barrier or an interface between a cytoplasm of the cell and an extracellular environment. A cell membrane typically includes a lipid bilayer along with other biological constituents, such as polypeptides, glycoproteins, lipoproteins, and polysaccharides.

As used herein, the terms “patient” and “subject” refer to a biological system from which a biological sample can be collected or to which a therapeutic agent can be administered. A patient can refer to a human patient or a non-human patient. Patients can include those that are healthy and those having a disease, such as cancer. Patients having a disease can include patients that have been diagnosed with the disease, patients that exhibit a set of symptoms associated with the disease, and patients that are progressing towards or are at risk of developing the disease.

As used herein, the term “biological sample” refers to a biological material that can be collected from a patient and used in connection with therapeutic agent selection and monitoring. Biological samples can include clinical samples, including body fluid samples, such as body cavity fluids, urinary fluids, cerebrospinal fluids, blood, and other liquid samples of biological origin; and tissue samples, such as biopsy samples, primary tumor samples, and other solid samples of biological origin. Biological samples can also include those that are manipulated in some way after their collection, such as by treatment with reagents, culturing, solubilization, enrichment for certain biological constituents, cultures or cells derived therefrom, and the progeny thereof.

As used herein, the term “therapeutic agent” refers to a treatment that can be administered to a patient, whether or not effective with respect to an intended purpose or target of the treatment. Therapeutic agents can include compounds of varying degrees of complexity that can influence a biological state, such as small molecules of therapeutic interest; naturally-occurring factors such as endocrine, paracrine, or autocrine factors or factors interacting with cell receptors of any type; intracellular factors such as those involved in intracellular signaling pathways; and factors isolated from other natural sources. Therapeutic agents can also include agents used in gene therapy, such as DNA and RNA. Also, antibodies, viruses, bacteria, and bioactive agents produced by bacteria and viruses can be considered as therapeutic agents. For certain applications, a therapeutic agent can include a composition including a set of active ingredients and a set of excipients.

As used herein, the terms “cancer,” “cancerous,” “malignancy,” “malignant,” and “tumor” refer to a disease in which certain cells exhibit relatively autonomous growth, so that the cells exhibit an aberrant growth phenotype characterized by a significant loss of control with respect to cell proliferation.

As used herein, the term “size” refers to a characteristic dimension. In the case of an object that is circular or spherical, a size of the object can refer to a diameter of the object, with the diameter being twice a radius of the object. In the case of an object having a non-uniform shape, a size of the object can refer to an average of various orthogonal dimensions of the object. Thus, for example, a size of an object that is elliptical or spheroidal can refer to an average of a major axis and a minor axis of the object. When referring to a set of objects as having a particular size, it is contemplated that the objects can have a distribution of sizes around that size. Thus, as used herein, a size of a set of objects can refer to a typical size of a distribution of sizes, such as a mean size, a median size, or a peak size.

### Nanomechanical Analysis System

Certain embodiments of this disclosure are directed to determining nanomechanical characteristics of cells, such as human cells. One example of a nanomechanical characteristic is the Young's modulus or the modulus of elasticity. The Young's modulus of a material is a measure of elasticity or stiffness of the material, and, typically, can be expressed as a ratio of an applied stress, such as an applied pressure, relative to a deformation or strain response within an elastic range of the material. If sufficient pressure is applied to a surface of a material, a displacement or movement of the surface typically occurs. If the applied pressure does not exceed an elastic limit of the material, the surface typically returns to its previous position once the applied pressure is removed. In the case of a cell, the Young's modulus can be at least partially influenced by the cytoskeleton of the cell. During malignant transformation, the cytoskeleton can be dynamically altered or remodeled, which, in turn, can lead to changes in the Young's modulus of the cell.

Another example of a nanomechanical characteristic is adhesiveness. The adhesiveness of a surface of a material is a measure of a tendency of the surface to attach or cling to another surface and, typically, can be expressed as an amount of force to detach or separate the surfaces once attached to one another. In the case of a cell, an adhesiveness of the cell can be at least partially influenced by biological constituents of a cell membrane. During malignant transformation, the nature or composition of the biological constituents can be dynamically altered or remodeled, which, in turn, can lead to changes in the adhesiveness of the cell.

According to some embodiments of this disclosure, a nanomechanical characteristic of a cell is determined by applying a stimulus or perturbation to the cell, detecting a response of the cell to the stimulus, and then deriving the nanomechanical characteristic of the cell based on its response. In some embodiments, a stimulus is applied by contacting a cell membrane with a probing element, and a resulting movement of the cell membrane is detected. Movement of the cell membrane can include lateral movement, vertical movement, stretching, contracting, or a combination thereof, and detection of the movement can be accomplished by implementing the probing element and associated components so as to be responsive to the movement. In turn, the nature and extent of the movement can be used to derive a nanomechanical characteristic of the cell, such as its Young's modulus. In other embodiments, a stimulus is applied by contacting a cell membrane with a probing element, and a resulting interaction of the cell membrane with the probing element is detected. Interaction of the cell membrane can include attachment to the probing element, rupturing of the cell membrane, or a combination thereof, and detection of the interaction can be accomplished by implementing the probing element and associated components so as to be responsive to the interaction. In turn, the nature and extent of the interaction can be used to calculate a nanomechanical characteristic of the cell, such as its adhesiveness.

In some embodiments, movement or interaction of a cell membrane is detected using an Atomic Force Microscope (AFM) operating in a contact mode. An AFM typically includes a spring element, such as a cantilever having one end adjacent to a cantilever body and another end adjacent to a probe or protrusion. The probe is elongated and extends along a direction substantially orthogonal to a lengthwise direction of the cantilever. A tip of the probe is positioned so as to be in contact with a cell, and serves as a mechanism for applying a stimulus to the cell. Movement of the cell membrane results in movement of the cantilever, such as in the form of deflection of the cantilever relative to a horizontal plane. Since a spring constant of the cantilever can be determined, an amount of pressure applied to the cell membrane can be determined based on the extent of deflection of the cantilever as a force is applied through the cantilever to the tip and, eventually, as pressure to the cell membrane. The force applied to the tip can be adjusted until a sufficient amount of pressure is applied to the cell membrane, and an elastic response of the cell is then determined. Similarly, attachment of the cell membrane to the tip results in movement of the cantilever, such as in the form of deflection of the cantilever relative to the horizontal plane. Since the spring constant of the cantilever can be determined, a detachment force to separate the tip from the cell membrane can be determined based on the extent of deflection of the cantilever as the tip is moved away from the cell. It will be appreciated that, while a spring element is sometimes referred to herein as a cantilever, the spring element can be implemented in a number of other ways, such as a coil spring, a torsion spring, or a leaf spring. Also, while a probing element is sometimes referred to herein as an AFM probe, the probing element is generally any elongated structure that can be used to apply a stimulus to a cell. Moreover, while some embodiments are described with reference to an AFM, it will be appreciated that other types of scanning probe microscopes or force-distance measuring devices can be used.

Desirably, an AFM tip is positioned adjacent to a central or nuclear region of a cell and is sized so as to allow determination of an inherent nanomechanical characteristic of the cell, with little or no influence from a substrate supporting the cell. If the tip is positioned near an edge of the cell or is sized beyond a certain extent, the tip can sometimes encounter resistance from the substrate before sufficiently engaging a cell membrane, thereby yielding a result that can differ from an inherent nanomechanical characteristic of the cell. Also, an AFM tip is desirably sized so as to allow determination of a local nanomechanical characteristic of a cell, rather than a corresponding whole cell or global characteristic, since the local nanomechanical characteristic can exhibit a greater correlation with respect to presence of a human disease or with respect to a particular type or subtype of the disease. Human cells of interest typically have sizes in the range of about 9 micrometer (μm) to about 30 μm, and associated nuclear regions typically have sizes in the range of about 3 μm to about 10 μm. Accordingly, for some embodiments, a radius of an AFM tip can be less than or equal to about 1 μm, such as from about 5 nanometer (nm) to about 900 nm, from about 5 nm to about 200 nm, from about 5 nm to about 100 nm, from about 5 nm to about 50 nm, from about 5 nm to about 20 nm, or below about 20 nm.

An AFM probe can be brought in contact with a cell membrane so as to apply a force in the range of about 1 pico-Newton (pN) to about 1 micro-Newton (μN), such as from about 10 pN to about 100 nano-Newton (nN) or from about 100 pN to about 10 nN. With a contact area on the cell membrane of a radius less than or equal to about 1 μm, an associated pressure applied to the cell membrane can be in the range of about 500 Pascal (Pa) up to about 6 kilo-Pascal (kPa) or more. A resulting extent of movement of the cell membrane can be in the range of about 0.1 nm to about 5 μm, such as from about 0.1 nm to about 3 μm, from about 0.1 nm to about 1 μm, from about 0.1 nm to about 500 nm, from about 1 nm to about 400 nm, from about 1 nm to about 300 nm, from about 1 nm to about 200 nm, or from about 1 nm to about 100 nm. Movement of the cell membrane can be detected with a single measurement or multiple measurements, which can occur at regular time intervals or irregular time intervals. For example, multiple measurements can occur at a frequency in the range of about 0.1 Hertz (Hz) to about 10 kilo-Hertz (kHz), such as from about 1 Hz to about 1 kHz, from about 1 Hz to about 100 Hz, or from about 1 Hz to about 10 Hz.

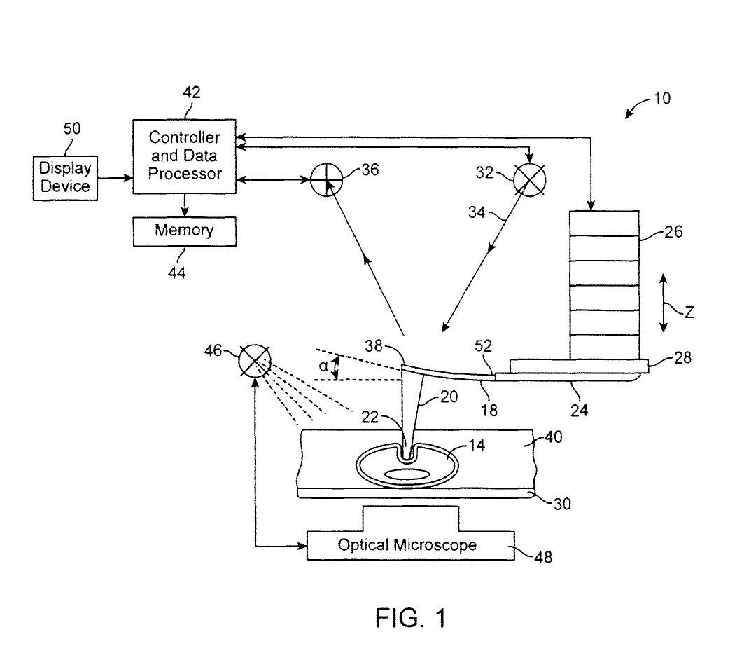

Attention turns to FIG. 1, which illustrates a nanomechanical analysis system 10 implemented in accordance with an embodiment of this disclosure. The system 10 includes a set of components corresponding to an AFM, which is used to determine a nanomechanical characteristic of a human cell 14 by way of a probing element. The cell 14 is placed within a fluid medium 40, which can be any cell culture medium, and is supported by a substrate 30. For reasons that are further described below, the substrate 30 is desirably formed of an optically transparent or translucent material, such as glass or plastic.

As illustrated in FIG. 1, the system 10 includes a cantilever 18 having one end 52 that is connected to a cantilever body 24, which is connected to a cantilever support 28. The cantilever support 28, in turn, is connected to an expansion element 26, such as a piezo-electric element, which is actuated to expand or contract so as to move the cantilever support 28 and other connected components vertically along the z-axis. Another end 38 of the cantilever 18 is adjacent to a probe 20 including a tip 22. The probe 20 is elongated and extends from a lower surface of the cantilever 18 in a direction substantially along the z-axis.

During operation of the system 10, the cantilever support 28 is moved downwardly as a result of actuating the expansion element 26. Eventually, the tip 22 of the probe 20 is brought in contact with the cell 14, and applies a force to the cell 14. As the expansion element 26 is expanded further, the force applied through the tip 22 increases and results in deformation of the cell 14. The cantilever 18 is flexible and has a relatively weak spring constant, and an elastic response from the cell 14 resists the applied force and results in deflection of the cantilever 18 by a certain angle a relative to a horizontal plane.

In the illustrated embodiment, the extent of deflection of the cantilever 18 is detected using a light source 32 and a photo-detector element 36. Referring to FIG. 1, the light source 32 is implemented as a laser, which emits a light beam 34 that is brought to focus on an upper surface of the cantilever 18. The light beam 34 is reflected towards and strikes the photo-detector element 36 as a laser spot. Deflection of the cantilever 18 moves the position of the laser spot with respect to the photo-detector element 36. As illustrated in FIG. 1, the photo-detector element 36 is implemented as an array of photo-detectors within four quadrants, and the photo-detectors produce outputs in response to the extent or presence of the laser spot within those quadrants. A difference in outputs between two or more quadrants indicates the position of the laser spot with respect to the photo-detector element 36 and, thus, the extent of deflection of the cantilever 18. Other mechanisms for detecting the deflection of the cantilever 18 are also contemplated. For example, bending of the cantilever 18 can be detected using an interference-detector element, which detects the extent of interference between a reflected light beam and an original light beam. As another example, a piezo-resistive element or a piezo-electric element can be included within or connected to the cantilever 18 so as to detect the extent of bending of the cantilever 18.

As illustrated in FIG. 1, the system 10 also includes a controller and data processor 42, which is connected to various components of the system 10 and serves to direct operation of those components. The controller and data processor 42 also processes outputs produced by the photo-detector element 36, and performs various data retrieval and manipulation operations. Referring to FIG. 1, the controller and data processor 42 is connected to a display device 50, which produces visual indications for a user of the system 10. The controller and data processor 42 is also connected to a memory 44, which stores computer code or executable instructions for performing various data retrieval and manipulation operations. The memory 44 also organizes data associated with these operations, such as within a database.

Still referring to FIG. 1, the system 10 further includes a light source 46 and an optical microscope 48, which is connected to the light source 46. The light source 46 illuminates the cell 14 from above, and the optical microscope 48 is implemented in an inverted configuration adjacent to a lower surface of the optically transparent or translucent substrate 30. Advantageously, the optical microscope 48 allows visual examination of the cell 14 through the substrate 30, and allows lateral positioning of the tip 22 over a central or nuclear region of the cell 14 with a desired level of precision. The optical microscope 48 also allows AFM analysis to be performed in conjunction with visual examination of the cell 14, such as for the purpose of locating and selecting the cell 14 for AFM analysis based on its morphological characteristics or its interaction with fluorescent labels.

While the single cell 14 is illustrated in FIG. 1, it is contemplated that multiple cells can be supported by the substrate 30 and can be subjected to similar analysis as described for the cell 14. In some instances, multiple human cells are prepared by subjecting a biological sample to a cytospin procedure. In such instances, a cell-counting device can be used to ensure that a sufficient number of cells are obtained in accordance with the cytospin procedure. For example, 2 to 100 living cells, such as 10 to 90 or 20 to 80 cells, can be subjected to AFM analysis, and the cells can be spread on the substrate 30 in a monolayer fashion.

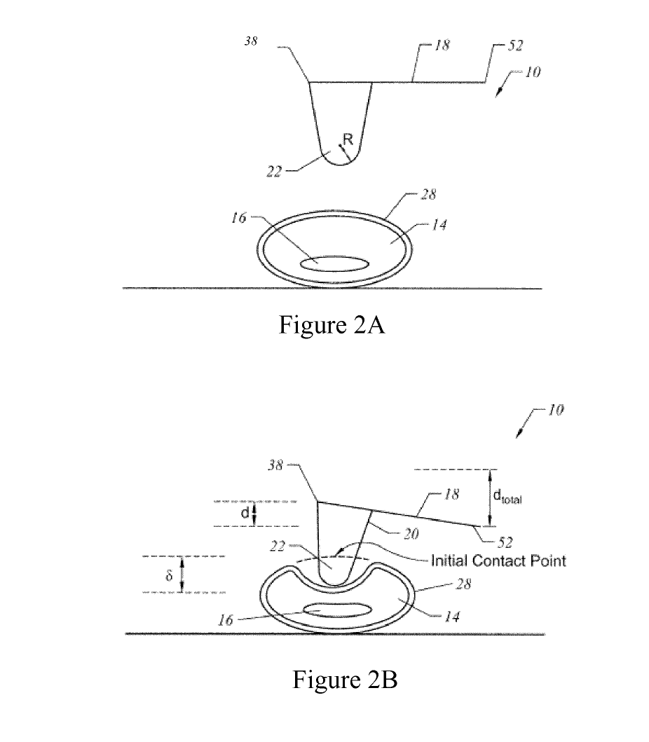

The operation of the system 10 can be further understood with reference to FIG. 2A and FIG. 2B. In particular, FIG. 2A illustrates the system 10 in a first configuration with the probe 20 positioned at a certain distance above the cell 14, while FIG. 2B illustrates the system 10 in a second configuration with the probe 20 in contact with the cell 14 and positioned over a nucleus 16 of the cell 14.

In the illustrated embodiment, the tip 22 of the probe 20 has a shape that is substantially a circular paraboloid, and, in the second configuration, the tip 22 has applied sufficient pressure to result in elastic deformation of a cell membrane 28. The Young's modulus E of the cell 14 can be calculated in accordance with the formula:

E=k(d)9/16 R^{-1/2} δ^{-3/2} (I)

where k is the spring constant of the cantilever 18, d is a deflection distance of the cantilever end 38, R is a radius of the tip 22, and δ is a deformation depth of the cell membrane 28. The deformation depth δ can be calculated in accordance with the formula:

δ=dₜₒₜₐₗ−d (II)

where dₜₒₜₐₗ is a distance that the cantilever end 52 has moved between the two configurations as a result of expansion of the expansion element 26 of FIG. 1. While the illustrated embodiment has been described with reference to a paraboloid tip shape, it is contemplated that the tip 22 can have various other shapes, and that the Young's modulus E can be similarly calculated for those shapes. Examples of other tip shapes include spherical shapes (e.g., associated with tips formed by connecting spheres to cantilevers), conical shapes, shapes associated with substantially flat or blunt tips, and shapes associated with tips that are curved or oblong (but not paraboloid).

### Nanomechanical Characteristics of Cells

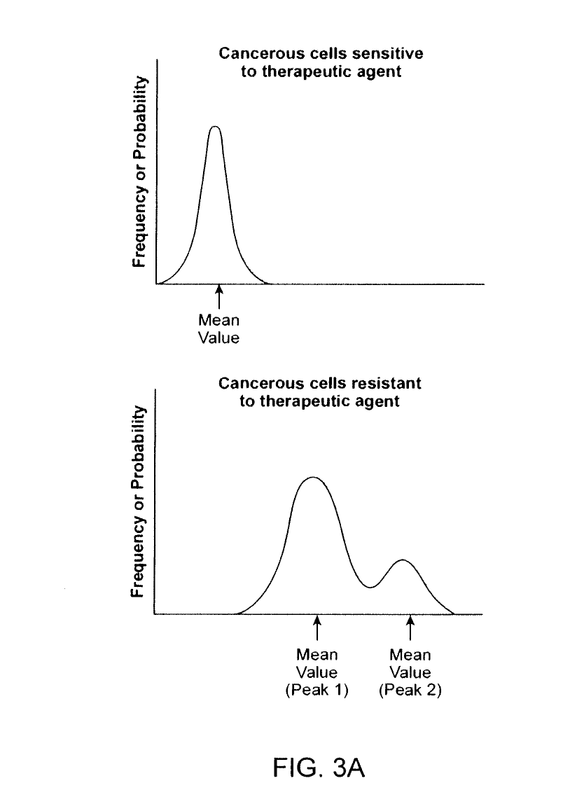

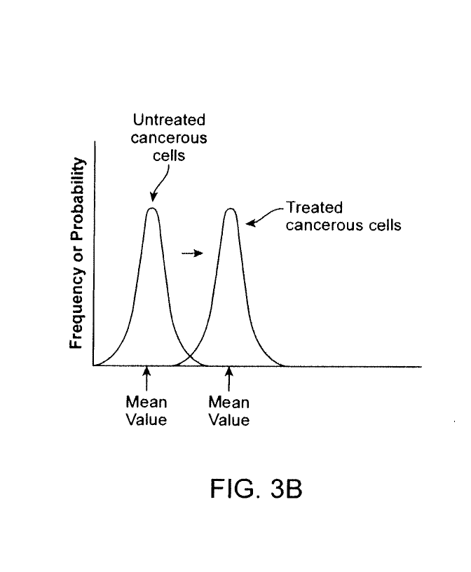

Attention next turns to FIG. 3A and FIG. 3B, which illustrate distributions of Young's modulus values characterized according to an embodiment of this disclosure.

FIG. 3A illustrates a distribution of Young's modulus values for a population of cancerous human cells that are sensitive to a therapeutic agent (top) and a distribution of Young's modulus values for a population of cancerous human cells that are resistant to the therapeutic agent (bottom).

Referring to FIG. 3A, certain notable differences between the distributions can be observed. In particular, the distribution of Young's modulus values for the therapeutic agent-sensitive cancerous cells substantially corresponds to a Gaussian distribution, while the distribution of Young's modulus values for the therapeutic agent-resistant cancerous cells substantially corresponds to a bimodal distribution with two peaks, labeled as peak 1 and peak 2. In the illustrated embodiment, the bimodal distribution for the therapeutic agent-resistant cancerous cells is represented as a combination of two Gaussian distributions corresponding to the two peaks. With respect to typical values of the distributions, mean Young's modulus values of the two peaks for the therapeutic agent-resistant cancerous cells are greater than a mean Young's modulus value for the therapeutic agent-sensitive cancerous cells, reflecting a reduced elasticity or an increased stiffness of the therapeutic agent-resistant cancerous cells relative to the therapeutic agent-sensitive cancerous cells. In the illustrated embodiment, the mean Young's modulus value of peak 1 for the therapeutic agent-resistant cancerous cells can be greater than the corresponding value for the therapeutic agent-sensitive cancerous cells by a factor of at least about 1.1, such as at least about 1.2 times greater, at least about 1.3 times greater, at least about 1.4 times greater, or at least about 1.5 times greater, and the mean Young's modulus value of peak 2 for the therapeutic agent-resistant cancerous cells can be greater than the corresponding value for the therapeutic agent-sensitive cancerous cells by a factor of at least about 2, such as at least about 2.2 times greater, at least about 2.4 times greater, at least about 2.6 times greater, or at least about 2.8 times greater.

Still referring to FIG. 3A, a spread in the distribution of Young's modulus values for the therapeutic agent-resistant cancerous cells is greater than a corresponding spread of Young's modulus values for the therapeutic agent-sensitive cancerous cells, reflecting an increased variability in elasticity or stiffness values for the therapeutic agent-resistant cancerous cells relative to the therapeutic agent-sensitive cancerous cells. In the illustrated embodiment, a standard deviation of Young's modulus values of each of peak 1 and peak 2 for the therapeutic agent-resistant cancerous cells can be greater than the corresponding value for the therapeutic agent-sensitive cancerous cells by a factor of at least about 1.5, such as at least about 1.6 times greater, at least about 1.7 times greater, at least about 1.8 times greater, at least about 1.9 times greater, or at least about 2 times greater.

FIG. 3B illustrates distributions of Young's modulus values for a population of therapeutic agent-sensitive cancerous human cells prior to administering a therapeutic agent (labeled as untreated) and a population of therapeutic agent-sensitive cancerous human cells subsequent to administering the therapeutic agent (labeled as treated). As illustrated in FIG. 3B, administering the therapeutic agent to the therapeutic agent-sensitive cancerous cells induces a shift in the distribution of Young's modulus values toward a reduced elasticity or an increased stiffness, as reflected by a greater mean or peak Young's modulus value subsequent to administering the therapeutic agent. The extent of the shift in the mean or the peak Young's modulus value can vary depending on a dosage and a frequency for which the therapeutic agent is administered, and, for example, the extent of the shift in the mean Young's modulus value can increase relative to an increase in the dosage of the therapeutic agent, an increase in the frequency for which the therapeutic agent is administered, or both. It is also contemplated that administering the therapeutic agent can induce a shift in the type of distribution of Young's modulus values, such as from a Gaussian distribution to a different type of probability distribution. By contrast, administering the therapeutic agent to therapeutic agent-resistant cancerous cells can induce little or no detectable change in distributions of Young's modulus values.

Although FIG. 3A and FIG. 3B represent distributions of Young's modulus values, distributions in values of another nanomechanical characteristic, such as adhesiveness, can be similarly characterized. Also, although a Gaussian distribution and a bimodal distribution are illustrated as specific examples of probability distributions, other types of probability distributions are contemplated, such as a log-normal distribution, a Pareto distribution, a trimodal distribution, and so forth.

### Analysis of Nanomechanical Characteristics of Cells

Referring to FIG. 3A, the distributions of Young's modulus values for the therapeutic agent-sensitive cancerous human cells and the therapeutic agent-resistant cancerous human cells can form the basis of nanomechanical assays for cancer therapy as well as development of therapeutic agents. In particular, the distributions illustrated in FIG. 3A can serve as reference nanomechanical profiles to which test nanomechanical profiles, such as determined for ex vivo human cells in clinical samples, can be compared as predictive biomarkers of sensitivity or resistance to a proposed therapeutic agent. Also, the distributions illustrated in FIG. 3A can serve as reference nanomechanical profiles to which test nanomechanical profiles, such as determined for cell lines or clinical samples, can be compared as predictive biomarkers of effectiveness of a therapeutic agent in development.

Advantageously, nanomechanical assays can be performed on a variety of clinical samples for prediction of effectiveness of a therapeutic agent for treating different types of cancer. For example, nanomechanical assays can be performed on body cavity fluids for treatment of metastatic adenocarcinoma. As another example, nanomechanical assays can be performed on urinary fluids for treatment of bladder cancer. As a further example, nanomechanical assays can be performed on primary tumor samples for treatment of breast cancer. Nanomechanical assays can be performed in conjunction with visual examination of human cells, such as in accordance with morphological examination or immunofluorescence labeling of the cells. Such visual examination can facilitate locating and selecting a subset of cells for nanomechanical assays based on morphological characteristics or interaction of the subset of cells with fluorescent labels.

According to an embodiment of this disclosure, a nanomechanical assay can be implemented as a diagnostic screen or test to predictive effectiveness of a proposed therapeutic agent to treat a particular type of cancer in a particular human patient. In particular, a clinical sample can be collected from the human patient, and multiple human cells in the clinical sample can be subjected to AFM analysis to determine respective test values of a nanomechanical characteristic, such as the Young's modulus. For example, measurements can be performed using the system 10 of FIG. 1 to determine a set of Young's modulus values for each cell of a group of cells in the clinical sample.

Next, test values of the nanomechanical characteristic resulting from AFM analysis can be subjected to statistical analysis to derive a test nanomechanical profile characterizing the nature or extent of a distribution of those test values. A comparison of the test nanomechanical profile can be performed with respect to either of, or both, a reference nanomechanical profile for therapeutic agent-sensitive cancerous cells and a reference nanomechanical profile for therapeutic agent-resistant cancerous cells, and results of the comparison can be indicative of whether cancerous cells of the human patient are predicted to be sensitive or resistant to the proposed therapeutic agent. In such manner, the effectiveness of the proposed therapeutic agent can be reliably predicted for the particular human patient.

For example, if Young's modulus values of cells in the clinical sample can be substantially fitted to a Gaussian distribution (e.g., a reference Gaussian distribution as illustrated in FIG. 3A (top)), a determination can be made that the cells are likely sensitive to the proposed therapeutic agent. Conversely, if the Young's modulus values can be substantially fitted to a bimodal distribution (e.g., a reference bimodal distribution as illustrated in FIG. 3A (bottom)), a determination can be made that the cells are likely resistant to the proposed therapeutic agent. As an alternative to, or in conjunction with, the manner of comparison described above, a mean and a spread in the Young's modulus values can be compared with means and spreads in reference values as illustrated in FIG. 3A. In particular, a determination can be made whether the cells are likely sensitive or resistant to the proposed therapeutic agent based on proximity of the mean or the spread in the Young's modulus values to a mean or a spread in reference values for therapeutic agent-sensitive cancerous cells or proximity of the mean or the spread in the Young's modulus values to a mean or a spread in reference values for therapeutic agent-resistant cancerous cells.

By providing reliable prediction of sensitivity or resistance to a therapeutic agent, an embodiment of a nanomechanical assay can be implemented for selection of a therapeutic agent suitable for a particular human patient, namely one for which cancerous cells of the human patient are predicted to be sensitive, rather than resistant. Examples of therapeutic agents that can be used to treat cancer include Green tea extract and various chemotherapy drugs such as cisplatin (or cis-diamminedichloridoplatinum(II)), paclitaxel, and taxane agents. In such manner, treatment can be tailored for the human patient, which can significantly improve recovery and survival rates.

Once a therapeutic agent is selected for a human patient, an embodiment of a nanomechanical assay can be implemented for monitoring effectiveness of the therapeutic agent in terms of impeding or reversing progression of cancer in the human patient. Monitoring effectiveness of the therapeutic agent also can be carried out to determine whether the human patient is developing resistance to the therapeutic agent, and, if so, whether another therapeutic agent should be selected for the human patient. The effectiveness of the therapeutic agent can be determined by administering the therapeutic agent to the human patient, collecting a clinical sample from the human patient subsequent to administering the therapeutic agent, and performing AFM analysis on the clinical sample in a similar manner as described above. The therapeutic agent can be administered in a variety of ways, such as orally, via inhalation, intravenously, or a combination thereof. Typically, a pharmaceutically effective dose of the therapeutic agent is administered to the human patient, and the effective dose can be determined using a variety of pharmacological techniques.

Next, post-treatment test values of a nanomechanical characteristic resulting from AFM analysis can be subjected to statistical analysis to derive a post-treatment nanomechanical profile characterizing the nature or extent of a distribution of those post-treatment test values. Baseline or pre-treatment test values of the nanomechanical characteristic can be determined by collecting a clinical sample from the human patient prior to administering the therapeutic agent, and performing AFM and statistical analysis on the clinical sample in a similar manner as described above to derive a baseline nanomechanical profile. The baseline nanomechanical profile can serve as a reference profile to which the post-treatment nanomechanical profile is compared, and results of the comparison can be indicative of whether the post-treatment nanomechanical profile is shifted toward the absence of cancer or toward cancer of a lesser degree of advancement, or is otherwise shifted in a manner indicative of effectiveness of the therapeutic agent. In some applications, either of, or both, a dosage and a frequency of administering the therapeutic agent can be adjusted or selected according to the comparison between the post-treatment nanomechanical profile and the baseline nanomechanical profile, such as based on an extent of shifting of a mean value or a peak value of the post-treatment nanomechanical profile. In other applications, the post-treatment nanomechanical profile can be compared with a reference nanomechanical profile for therapeutic agent-resistant cancerous cells, and results of the comparison can be indicative of whether the human patient is developing resistance to the therapeutic agent.

### EXAMPLE

The following example describes specific aspects of some embodiments of this disclosure to illustrate and provide a description for those of ordinary skill in the art. The example should not be construed as limiting this disclosure, as the example merely provides specific methodology useful in understanding and practicing some embodiments of this disclosure.

### Correlative Nanomechanical Profiling with Super-Resolution F-Actin Imaging to Elucidate Mechanisms of Cisplatin Resistance in Ovarian Cancer Cells

As set forth in this example, biomechanics of cisplatin-treated ovarian cancer cells were measured quantitatively at a nanoscale level using an AFM. This example demonstrates that cisplatin modulates the cellular nanomechanics of ovarian cancer cells. Sensitive cells show dose-dependent increase in cell stiffness, which is effected by disrupting the F-actin polymerization. In contrast, resistant cells show no significant changes in cell stiffness upon cisplatin treatment. Further, stimulated emission depletion (STED), a super-resolution light microscopy technique, shows that, at the molecular level, F-actin is remodeled considerably in cisplatin-sensitive and cisplatin-resistant cells. These findings reveal a role of the actin remodeling mechanism in cisplatin resistance of ovarian cancer cells, demonstrating applications of nanomechanical profiling as a biomarker for cancer drug sensitivity.

Cellular nanomechanical profiles were measured to explore the structural and molecular mechanisms of cisplatin resistance in preclinical models of ovarian cancer. OVCAR5 is a human epithelial carcinoma cell line of the ovary, which is established from the ascitic fluid of a patient with progressive ovarian adenocarcinoma without prior cytotoxic treatment and is used as a model for studying cisplatin drug sensitivity. An AFM was used to detect the nanoscale differences in cell stiffness of cisplatin-sensitive (OVCAR5) and cisplatin-resistant cell lines (OVCAR5-CisR). Additionally, correlation of AFM data was conducted with STED, which allows subdiffraction limit visualization of cellular structures, at the molecular level, with nanometer precision. To understand the molecular basis of changes in cellular biomechanics associated with cisplatin treatment, in particular cellular cytoskeleton, the F-actin structure and organization were probed at a high resolution (−100 nm) using STED microscopy, which allows visualization of the dense cellular actin network. Correlative AFM-based cellular nanomechanical profiling and molecular-level actin cytoskeleton imaging via STED microscopy provide high-resolution details on the influence of cisplatin treatment on the structural-mechanical properties of ovarian cancer cells. Actin remodeling is proposed as a cisplatin drug resistance mechanism at the single-cell and subcellular levels.

Methods:

Human Ovarian Cancer Cell Lines

SKOV3 (American Type Culture Collection; Manassas, Va.) and OVCAR5 cells were maintained in Dulbecco's Modified Eagle's Medium supplemented with about 10% fetal bovine serum. SKOV3-CisR and OVCAR5-CisR cells were established by exposing the parental SKOV3 and OVCAR5 cell lines, respectively, to increasing concentrations of cisplatin over 12 months. OVCAR5-CisR cells are about 20- and SKOV3-CisR cells are about 8-fold more resistant to cisplatin compared to their syngeneic cisplatin sensitive counterparts. Cells were grown at about 37° C. in a humidified atmosphere containing about 5% CO₂ and about 95% air.

Drug Treatments

Cells were treated with about 1 or about 5 μg/mL cisplatin (Sigma-Aldrich, St. Louis, Mo.) for about 24 hours before AFM measurements or actin staining In cytoskeleton depolymerizing experiments, about 10 μM cytochalasin D was added either about 1 hour before cisplatin treatment or during the last hour of cisplatin treatment.

AFM Measurements

A protocol was established to exclude unhealthy or apoptotic cells from measurements by staining cells with AnnexinV (Sigma-Aldrich) as an indicator of apoptosis. Stained cells are dead or apoptotic, and typically float away and are no longer attached to a surface. Cells that stained positive for AnnexinV were excluded from AFM analysis. Measurements were conducted using a Catalyst AFM (Bruker Instruments, Santa Barbara, Calif.) with a combined inverted optical/confocal microscope (Zeiss Corp., Thornwood, N.Y.). This combination permits lateral positioning of an AFM tip over the cell center with submicron precision. AFM images and mechanical measurements were collected in contact mode using sharpened silicon nitride cantilevers with experimentally determined spring constants of about 0.02 N/m and a tip radius of less than about 20 nm. Mechanical measurements were obtained at about 37° C. with force measurements recorded at a pulling rate of about 1 Hz. Force-displacement curves were recorded for determination of Young's modulus values. Conversion of force-displacement curves to force-indentation curves allows the determination of the local cell surface elasticity or stiffness (Young's modulus, E). The measurement outcomes include averaged E values. Nanoscope files were converted to Igor. 6.0 (Wavemetrix) and used to generate two-dimensional (2D) stiffness matrix of the cell populations, allowing more precise determination of localized cell stiffness. AFM measurements were obtained in at least 60 cells in three different experiments. The AFM tip was precisely positioned (within micron range) on top of the nucleus using motorized stage and inverted optical view of the combined confocal-AFM microscope.

STED Microscopy

A Leica TCS SP5 STED confocal system (Leica Micro-systems, Wetzlar, Germany) equipped with a 640-nm pulsed diode laser (PicoQuant, Berlin, Germany) was used for excitation combined with a pulsed ultrafast Ti:sapphire infrared laser (Mai Tai Broadband; Spectra-Physics, Santa Clara, Calif.), fully tunable from about 710 nm to about 990 nm. Cells were fixed and actin stained with phalloidin-Atto 647N (ATTO-TEC, Siegen, Germany). Images were filtered and flattened using SPIP 5.8 Image Metrology (Horsholm, Denmark).

Statistical Significance

Data were expressed as mean±standard error of mean (SEM). Statistical significance was identified by Student's t-test or one-way analysis of variance (Origin 8.0, OriginLab Corporation, Northmpton, Mass.) for the difference among treated pairs.

A P value<0.05 was considered to be statistically significant.

Results:

Cisplatin-Sensitive and Cisplatin-Resistant Human Ovarian Cancer Cells Possess Distinct Cell Stiffness (Young'S Modulus)

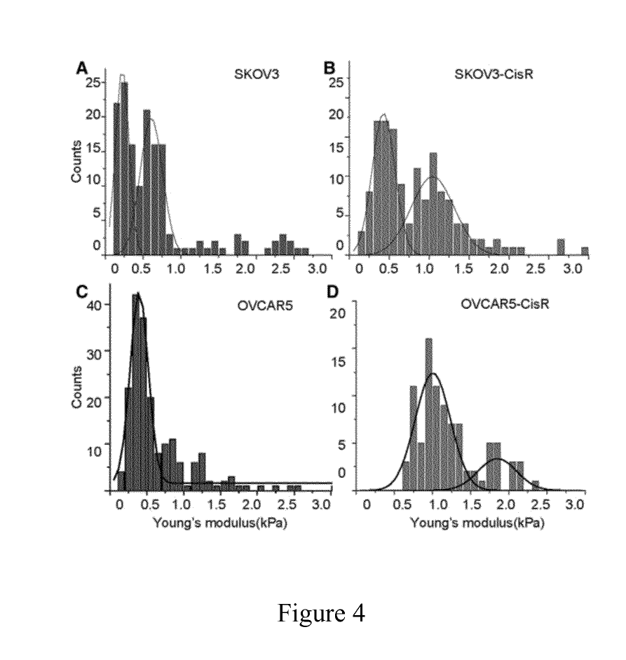

To evaluate the effect of cisplatin sensitivity on the properties of ovarian cancer, pairs of cisplatin-resistant human ovarian cancer cell lines were generated as described in the Methods section. The cisplatin-sensitive human ovarian cancer cell lines SKOV3 and OVCAR5 were incubated in the presence of increasing concentrations of cisplatin over several months, resulting in SKOV3-CisR and OVCAR5-CisR cells that were 8- and 20-fold more resistant to cisplatin compared to their syngeneic cisplatin sensitive counterparts. Cell stiffness for both pairs of cisplatin-sensitive and resistant cell lines, SKOV3/SKOV3-CisR and OVCAR5/OVCAR-CisR, was assessed based on AFM Young's modulus analysis. Cisplatin-resistant cell lines (SKOV3-CisR and OVCAR5-CisR) both show a significantly higher cell stiffness (P<0.05) compared to their cisplatin-sensitive counterparts (SKOV3 and OVCAR5) (see FIG. 4). Young's modulus for SKOV3 and OVCAR5 cells displayed a Gaussian distribution, whereas SKOV3-CisR and OVCAR5-CisR cells displayed a bimodal distribution with two main peaks. The average E values (mean±SEM) for SKOV3 and OVCAR5 cells were 0.41±0.04 kPa and 0.64±0.03 kPa, respectively.

The resistant cell lines showed bimodal distribution and higher cell stiffness. E values (mean±SEM) for SKOV3-CisR were 0.42±0.01 kPa (peak 1) and 1.04±0.03 kPa (peak 2). E values (mean±SEM) for OVCAR5-CisR were 1.0±0.03 kPa (peak 1) and 1.84±0.1 kPa (peak 2), respectively. The OVCAR5 cell lines were selected to further elucidate the nanomechanical profiles for platinum-sensitive compared to platinum-resistant ovarian cancer cells.

OVCAR5 Cells Show Cisplatin Dose-Dependent Increase in Cell Stiffness

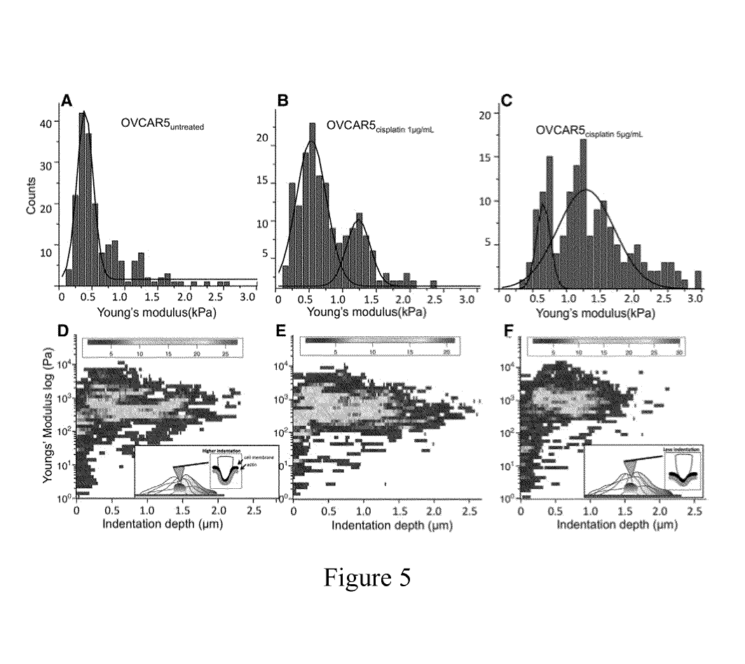

Analysis was next conducted to determine whether the changes observed in cell mechanical stiffness in cisplatin-resistant cells can be induced using short-term treatment of platinum-sensitive cells with cisplatin. Cisplatin-sensitive OVCAR5 cells (E=0.64±0.03 kPa) were incubated with about 1 and about 5 μg/mL cisplatin and subjected to AFM measurements after about 24 hours of treatment. Measurements were obtained over more than 60 isolated live, nonapoptotic cells in three independent experiments (see FIG. 5). The E value (mean±SEM) was observed to increase in OVCAR5 cells, resulting in E=0.53±0.01 kPa (peak 1) and 1.23±0.04 kPa (peak 2) after about 1 μg/mL cisplatin treatment. Further increase was observed with E=0.65±0.01 kPa (peak 1) and 1.28±0.06 kPa (peak 2) after about 5 μg/mL cisplatin treatment of cells in vitro.

In addition to the Young's modulus of the cell structure, the localized stiffness of cells was measured as a function of AFM probe indentation or deformation depth. The 2D maps of stiffness versus indentation matrix (see FIG. 5, D-F) obtained for OVCAR5 cells show a distinct shift in peak indentation depth after cisplatin treatment with values ranging from 1.2±0.04 μm (OCVAR5, FIG. 5, D) to 0.7±0.03 μm (OCVAR5_{cisplatin 1 μg/mL}, FIG. 5, E) and 0.5±0.03 μm (OCVAR5_{cisplatin 5 μg/mL}, FIG. 5, F). Schematics of mechanical compliance of cells under different conditions are shown in insets in FIGS. 5, D and E. The transition of the peak indentation depth, where higher values suggest greater deformation or lower stiffness under constant applied force, indicates the differences in the biomechanical profiles of OVCAR5 cells as a result of cisplatin treatment.

OVCAR5-CisR Cells Stiffness is Not Noticeably Altered Upon Cisplatin Treatment

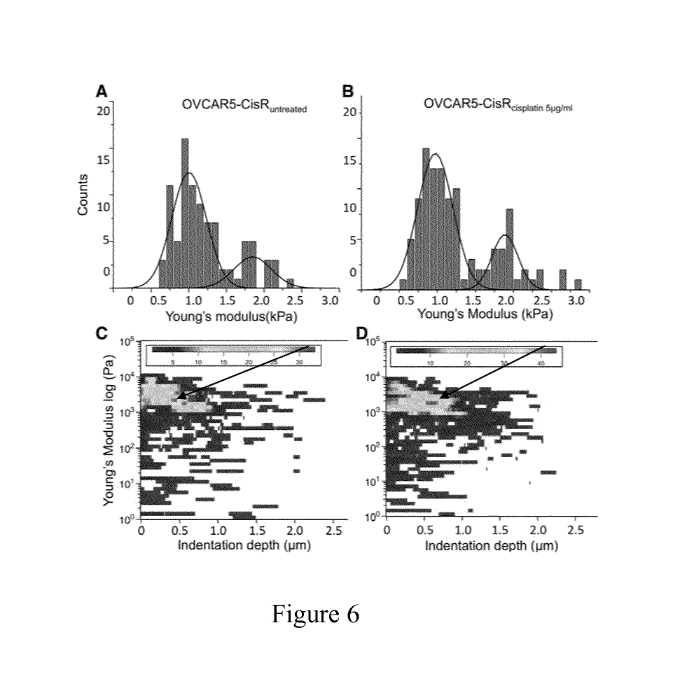

Analysis was next conducted to determine whether short-term treatment with cisplatin affects the cell stiffness of OVCAR5-CisR cells. As shown in FIG. 6, after cisplatin treatment, OCVAR5-CisR_{cisplatin 5 μg/mL} cells with E=0.97±0.01 kPa (peak 1) and 1.88±0.05 kPa (peak 2) (FIG. 6, A, B) show no significant difference (P=0.45) between untreated and cisplatin-treated cells. The 2D stiffness versus indentation matrix map for OVCAR5-CisR cells (FIG. 6, C) shows more localized peak indentation distribution (0.5±0.06 μm; FIG. 6, C) compared to OVCAR5 cells. The localized peak indentation remains largely unchanged after cisplatin treatment (0.5±0.04 μm for OCVAR5-CisR_{cisplatin 5 μg/mL}; FIG. 6, D).

Cisplatin-Induced Increase in OVCAR5 Cell Stiffness is Mediated Via Actin Cytoskeleton Modification

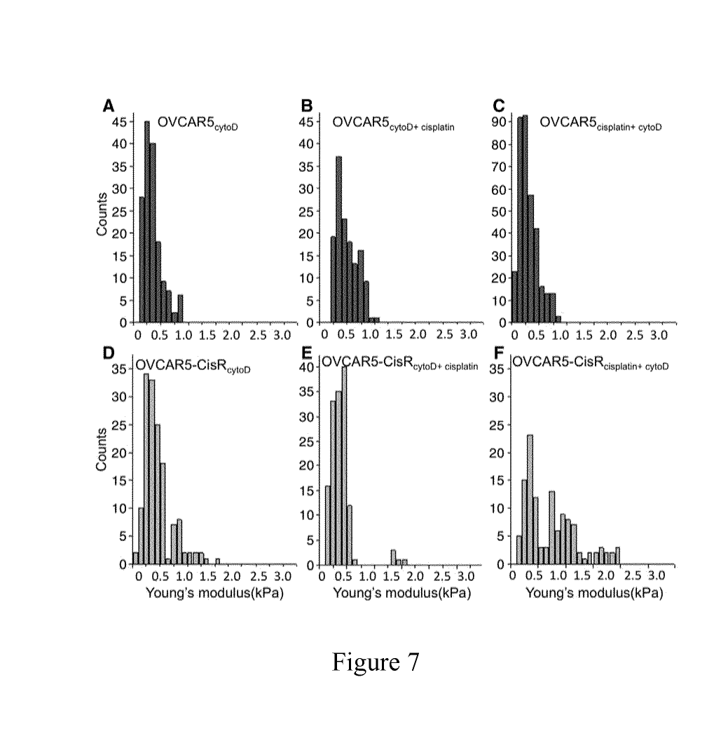

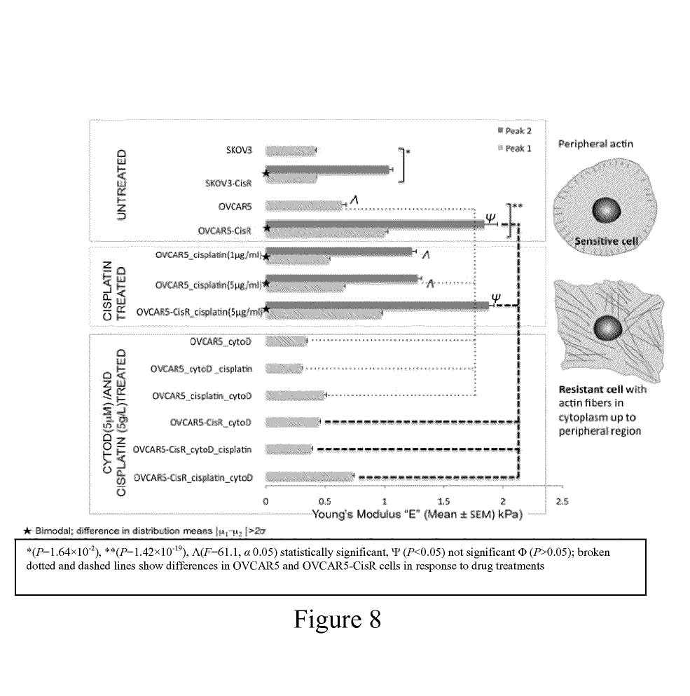

The elastic properties of living cells are determined (at least in part) by the cellular actin cytoskeleton. If a functional actin cytoskeleton is involved to induce an increase in cell stiffness by cisplatin, it is expected that this increase is absent once the actin cytoskeleton is disrupted. To test whether the actin cytoskeleton mediates the observed cisplatin-induced stiffness increase, the stiffness of OVCAR5 and OVCAR5-CisR cells exposed to cisplatin was measured, after treatment with about 5 μM cytochalasin D, an inhibitor of actin polymerization (FIG. 7). The results are summarized in FIG. 8. As expected, treatment with cytochalasin D alone results in marked decrease in the stiffness of both OVCAR5 (OVCAR5_{cytoD} E=0.34±0.02 kPa) (FIG. 7, A) and OVCAR5-CisR (OVCAR5-CisR_{cytoD} E=0.45±0.01 kPa) (FIG. 7, D) cells. Cisplatin treatment alone results in increase in cell stiffness of OVCAR5 cells (FIG. 5, C); however, when the OVCAR5 cells were first exposed to cytochalasin D and then to cisplatin, no noticeable increase in the stiffness of OVCAR5 was observed (OVCAR5_{cytoD+cisplatin} E=0.30±0.02 kPa) (FIG. 7, B). Although cisplatin treatment alone did not increase OVCAR5-CisR cell stiffness (FIG. 5, B), OVCAR5-CisR cells first exposed to cytochalasin D and then to cisplatin show reduced stiffness (OVCAR5-CisR_{cytoD+cisplatin} E=0.38±0.01 kPa) (FIG. 7, E). Further, the cells were treated initially with cisplatin and subsequently with cytochalasin D. Both OVCAR5 (OVCAR5_{cisplatin+cytoD} E=0.49±0.01 kPa) (FIG. 7, C) and OVCAR5-CisR (OVCAR5-CisR_{cisplatin+cytoD} E=0.73±0.05 kPa) (FIG. 7, F) show significant decline in cell stiffness (P<0.05), although a smaller decline than treatment with cytochalasin D followed by cisplatin (see FIG. 8). Thus, cisplatin failed to noticeably increase stiffness of OVCAR5 and OVCAR5-CisR cells once the actin cytoskeleton was disrupted. The results indicate that cell stiffness involves actin polymerization and indicate that cell stiffness increase upon cisplatin treatment is at least partly due to dynamic changes in the cellular actin cytoskeleton.

Nanostructural Organization of Cytoskeletal Actin Revealed Via Super-Resolution STED Microscopy

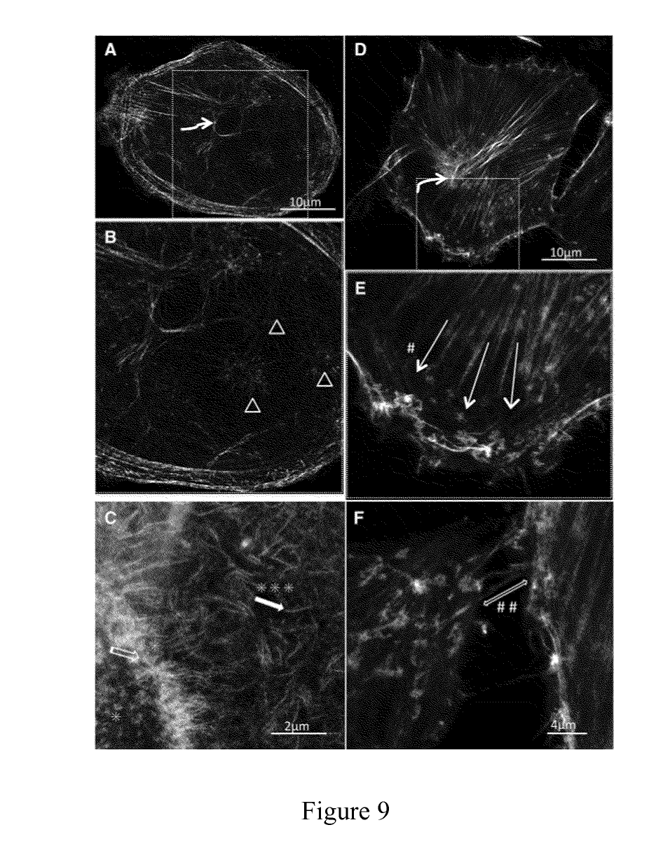

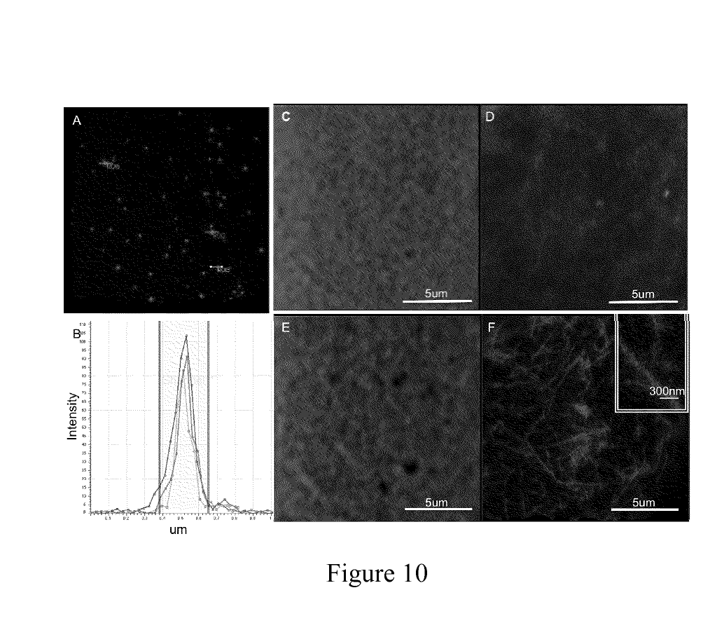

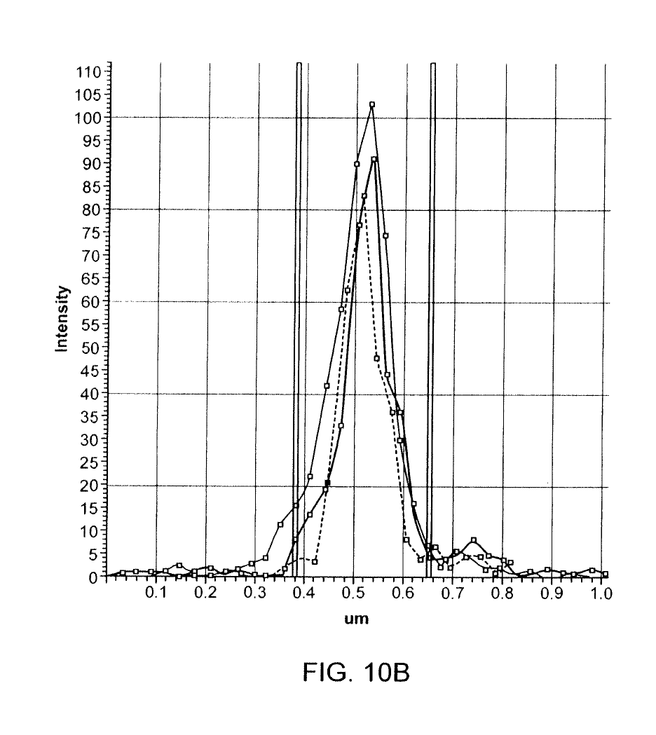

The role of cellular cytoskeletal protein, in particular actin remodeling, was characterized and correlated with the molecular mechanism associated with nanomechanical changes in cisplatin-treated cells using high-resolution STED microscopy (about 70-nm resolution). STED is an imaging technique that allows imaging beyond the classic diffraction limit. Using STED imaging, the structural organization of F-actin was visualized at the filament level (see FIG. 9) with spatial resolution of about 100 nm. FIG. 10 demonstrates the capability of STED to resolve details of the F-actin filament architecture (arrows), such as closely arranged short F-actin bundles surrounding the cell nucleus, not typically resolved via confocal imaging. Actin polymerization and depolymerization occur dynamically within cells in response to either, or both, chemical and mechanical stresses. Imaging the F-actin organization provides structural insights into the actin remodeling mechanisms likely to be associated with development of cisplatin drug resistance in ovarian cancer cells (see FIG. 9).

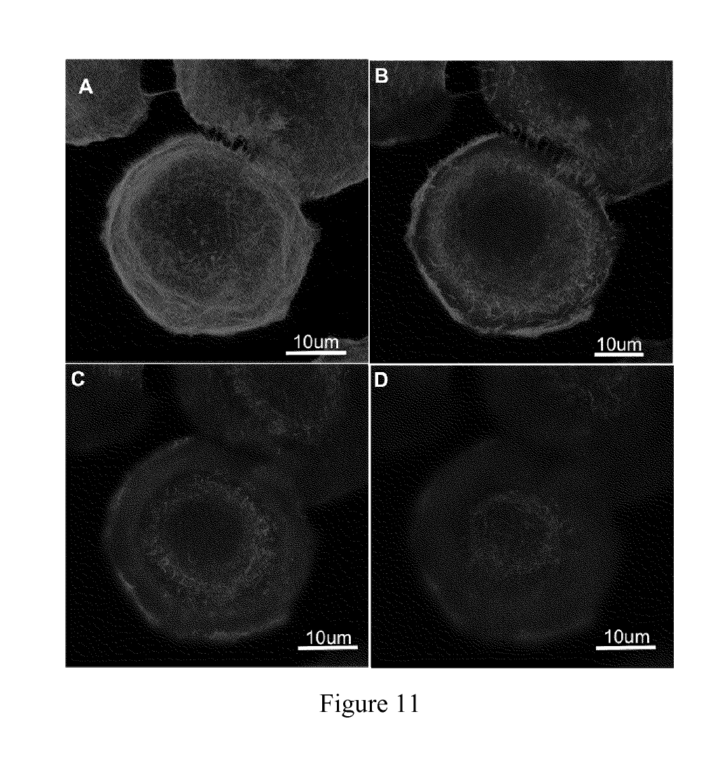

Significant differences were observed in the cellular F-actin cytoskeleton of ovarian cancer cells varying in cisplatin sensitivity (FIG. 9, A-C). A noteworthy feature of the OVCAR5 cell cytoskeleton is the apparent aggregation of the short F-actin filaments in numerous small clusters (FIG. 9, B) scattered throughout the cytoplasm. Such short, randomly aligned filaments or aggregates (FIG. 9, C) exist substantially throughout the entire volume of the cell and around the cell nucleus (see FIG. 11). However, in contrast, in the case of OVCAR5-CisR cells (FIG. 9, D-F), dense, radially aligned actin filaments (stress fibers, long, straight bundles of actin filaments) were observed. The filaments extend across the nucleus into the cytoplasm and up to the cell membrane as an intricate, densely organized and structurally oriented network (FIG. 9, D, E). The dense networks of actin filaments were localized at the base of the nucleus in OVCAR5-CisR cells. However, little or no stress fibers were observed in the case of the OVCAR5 cell nucleus. Both cell types were imaged (10 cells in different areas of the sample) on two different days, and the images represent typical actin cytoskeleton structures observed for each cell type. Additionally, OVCAR5-CisR cells show several actin dense cell-cell contacts as shown in FIG. 9, F.

Discussion:

The analysis has identified mechanical and actin cytoskeletal remodeling characteristics in cisplatin-sensitive and resistant human ovarian cancer cell lines using an AFM. The results demonstrate that cisplatin modulates the cellular nanomechanics of human ovarian cancer cells. Sensitive cells show dose-dependent increase in cell stiffness, which is affected by disrupting the F-actin via cytochalasin D treatment. In contrast, resistant cells show no significant changes in cell stiffness upon cisplatin treatment. Further, using super-resolution STED microscopy, it is demonstrated that, at the molecular level, F-actin is remodeled considerably in cisplatin-sensitive and cisplatin-resistant cells.

Implications of Cisplatin-Induced Cell Stiffening and Resistance Development

The results reveal that ovarian cancer cells differing in drug sensitivity to cisplatin show characteristically different cell stiffness profiles (see FIG. 8), indicating that cell mechanics can play a role in drug resistance. Short-term (24 hours) cisplatin treatment induces an increase in the stiffness of OVCAR5 cells in a concentration-dependent manner. Cisplatin-resistant cells showed a bimodal distribution, with two peaks in the histogram of cell stiffness, and with the E values of peak 1 similar to those of sensitive cells. Such observed bimodal behavior indicates the presence of two differing subpopulations within the cisplatin-resistant cells. Several multifactorial molecular mechanisms localized within the cell membrane, cytoplasm, or nucleus (and influenced by factors like membrane fluidity, cisplatin uptake, and molecular signaling pathways) may result in resistant cell phenotypes. However, changes in elastic properties of living cells are associated (at least in part) with remodeling of the cellular actin cytoskeleton, as observed in the case of cisplatin-resistant cells. Cisplatin induced a concentration-dependent increase in stiffness of OVCAR5 cells. The sensitive cells when treated with cisplatin showed an increase in cell stiffness and attained a cell stiffness profile similar to that of the untreated resistant cells. The data show that nanomechanical variations are correlated with cell in vitro drug sensitivity.

The actin network is a structural and functional system that provides the basic infrastructure for maintaining cell morphology and functions such as adhesion, motility, exocytosis, endocytosis, and cell division. Actin remodeling has a role in regulating the morphological and phenotypical events of a malignant cell, and may reflect the activation of oncogenic actin signaling pathways (e.g., Ras and Src) or the inactivation of actin-binding proteins that have tumor suppressor functions (e.g., gelsolin or E-cadherins). The data set forth in this example show that disrupting the actin cytoskeleton in sensitive cells results in lowering the cell stiffness-inducing effect of cisplatin, and indicate that actin remodeling has a role in cisplatin-mediated cell stiffness modulation.

At the molecular level, STED microscopy reveals significant differences in the cellular F-actin cytoskeleton of ovarian cancer cells varying in cisplatin sensitivity. The structural insights into the actin remodeling in ovarian cancer cells, such as randomly oriented actin filaments and lack of actin stress fibers in cisplatin-sensitive cells (FIG. 8, upper inset), but highly organized actin architecture in cisplatin-resistant cells (FIG. 8, lower inset), indicate a role of actin modulation in platinum chemotherapy resistance in ovarian cancer cells.

In summary, this example demonstrates that quantitative biomechanical variations in ovarian cancer cells are related to cisplatin drug resistance, which are mediated (at least in part) via actin remodeling. Cisplatin modulates the cellular nanomechanics of human ovarian cancer cells, and sensitive cells show a dose-dependent increase in cell stiffness, which is affected by disrupting the F-actin via cytochalasin D treatment. At the molecular level, STED microscopy reveals that F-actin is remodeled considerably in cisplatin-sensitive and cisplatin-resistant cells. The results indicate an actin remodeling mechanism in cisplatin resistance of ovarian cancer cells. Additionally, the capability to detect and correlate the cell biomechanical characteristics quantitatively at the single-cell level using AFM and super-resolution STED microscopy provides a combined nanomechanical and nanostructural insight into the cancer drug sensitization problem.

A practitioner of ordinary skill in the art should require no additional explanation in developing the embodiments described herein, but may nonetheless find some helpful guidance by examining PCT Publication No. WO 2009/142661, the disclosure of which is incorporated herein by reference in its entirety.

Certain embodiments of this disclosure relate to a computer storage product with a non-transitory computer-readable storage medium including data structures and computer code for performing a set of computer-implemented operations. The medium and computer code can be those specially designed and constructed for the purposes of embodiments of this disclosure, or they can be of the kind well known and available to those having ordinary skill in the computer software arts. Examples of computer-readable storage media include: magnetic media such as hard disks, floppy disks, and magnetic tape; optical media such as Compact Disc-Read Only Memories (“CD-ROMs”) and holographic devices; magneto-optical media such as floptical disks; and hardware devices that are specially configured to store and execute computer code, such as Application-Specific Integrated Circuits (“ASICs”), Programmable Logic Devices (“PLDs”), Read Only Memory (“ROM”) devices, and Random Access Memory (“RAM”) devices. Examples of computer code include machine code, such as produced by a compiler, and files including higher-level code that are executed by a computer using an interpreter. For example, an embodiment of this disclosure can be implemented using Java, C++, or other object-oriented programming language and development tools. Additional examples of computer code include encrypted code and compressed code. Another embodiment of this disclosure can be implemented in hardwired circuitry in place of, or in combination with, computer code.

While the invention has been described with reference to the specific embodiments thereof, it should be understood by those skilled in the art that various changes may be made and equivalents may be substituted without departing from the true spirit and scope of the invention as defined by the appended claims. In addition, many modifications may be made to adapt a particular situation, material, composition of matter, method, operation or operations, to the objective, spirit and scope of the invention. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the invention. Accordingly, unless specifically indicated herein, the order and grouping of the operations is not a limitation of the invention.

What is claimed is:

1. A method of treating a patient having cancer, comprising:
providing a biological sample from the patient, the biological sample including multiple cancerous cells;
detecting a response of the biological sample to a probing element;
based on the response, determining test values for the biological sample, the test values being indicative of a nanomechanical characteristic of the cancerous cells;
deriving a test nanomechanical profile characterizing a distribution of the test values; and
based on the test nanomechanical profile, selecting a therapeutic agent to treat the patient.

2. The method of claim 1, wherein detecting the response of the biological sample is performed using an Atomic Force Microscope including the probing element.

3. The method of claim 1, wherein detecting the response of the biological sample includes:
contacting a cell membrane of at least one of the cancerous cells with the probing element; and
detecting movement of the cell membrane.

4. The method of claim 1, wherein the nanomechanical characteristic corresponds to the Young's modulus of the cancerous cells.

5. The method of claim 1, wherein selecting the therapeutic agent is performed if the test nanomechanical profile is indicative of sensitivity of the cancerous cells to the therapeutic agent.

6. The method of claim 1, wherein selecting the therapeutic agent includes comparing the test nanomechanical profile with a reference nanomechanical profile indicative of sensitivity to the therapeutic agent.

7. The method of claim 1, wherein selecting the therapeutic agent includes comparing the test nanomechanical profile with a reference nanomechanical profile indicative of resistance to the therapeutic agent.

8. The method of claim 1, wherein deriving the test nanomechanical profile includes fitting the test values to at least one of a Gaussian distribution and a bimodal distribution.

9. The method of claim 1, further comprising administering the selected therapeutic agent to the patient.

10. A method of treating a patient having cancer, comprising:
providing a first biological sample from the patient prior to administering a therapeutic agent;
deriving a baseline nanomechanical profile characterizing a distribution of Young's modulus values of a first set of cancerous cells in the first biological sample;
providing a second biological sample from the patient subsequent to administering the therapeutic agent;
deriving a post-treatment nanomechanical profile characterizing a distribution of Young's modulus values of a second set of cancerous cells in the second biological sample; and
based on a comparison between the post-treatment nanomechanical profile and the baseline nanomechanical profile, adjusting administering of the therapeutic agent to the patient.

11. The method of claim 10, wherein adjusting administering of the therapeutic agent includes determining an extent of shifting of the post-treatment nanomechanical profile away from the baseline nanomechanical profile.

12. The method of claim 10, wherein adjusting administering of the therapeutic agent includes adjusting at least one of a dosage and a frequency of administering the therapeutic agent.

13. A non-transitory computer-readable storage medium to monitor treatment of a human patient having cancer, comprising executable instructions to:
derive a baseline nanomechanical profile characterizing a distribution of Young's modulus values of a first set of cancerous cells, the first set of cancerous cells being collected from the human patient prior to administering a therapeutic agent;
derive a post-treatment nanomechanical profile characterizing a distribution of Young's modulus values of a second set of cancerous cells, the second set of cancerous cells being collected from the human patient subsequent to administering the therapeutic agent; and
based on a comparison between the post-treatment nanomechanical profile and the baseline nanomechanical profile, produce an indication of effectiveness of the therapeutic agent for the human patient.

14. The non-transitory computer-readable storage medium of claim 13, wherein the executable instructions to produce the indication of effectiveness include executable instructions to determine whether the post-treatment nanomechanical profile is shifted towards greater Young's modulus values, relative to the baseline nanomechanical profile.

15. The non-transitory computer-readable storage medium of claim 13, wherein the executable instructions to produce the indication of effectiveness include executable instructions to determine whether a mean value of the post-treatment nanomechanical profile is shifted away from a mean value of the baseline nanomechanical profile.
